(19) United States
(12) Patent Application Publication
(10) Pub. No.: US 2005/0234438 A1
(43) Pub. Date: Oct. 20, 2005

(54) Ultrasound medical treatment system and method

(76) Inventors: T. Douglas Mast, Cincinnati, OH (US); Waseem Faidi, Clifton Park, NY (US); Inder Raj S. Makin, Loveland, OH (US); Megan M. Runk, Cincinnati, OH (US); Peter G. Barthe, Phoenix, AZ (US); Michael H. Slayton, Tempe, AZ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/825,092

(22) Filed: Apr. 15, 2004

(51) Int. Cl. A61B 18/04

(52) U.S. Cl. 606/27

(57) ABSTRACT

An embodiment of an ultrasound medical treatment system includes an ultrasound medical-treatment transducer and a controller. The controller controls the medical-treatment transducer to emit ultrasound to thermally ablate patient tissue. The control includes a control parameter. The controller changes the control parameter based on receiving an indication of an occurrence in the patient tissue of a transient, ultrasound-caused, ultrasound-attenuating effect. A method for medically treating patient tissue with ultrasound includes controlling the medical-treatment transducer with the control parameter set to a first setting, receiving an indication of the occurrence of the ultrasound-attenuating effect, changing the control parameter to a second setting based on receiving the indication, and controlling the medical-treatment transducer with the control parameter set to the second setting.

### FIELD OF THE INVENTION

The present invention relates generally to ultrasound, and more particularly to an ultrasound medical treatment system and method.

### BACKGROUND OF THE INVENTION

Known ultrasound medical-treatment systems and methods include using ultrasound imaging (at low power) of patients to identify patient tissue for medical treatment and include using ultrasound (at high power) to ablate identified patient tissue by heating the tissue. In one arrangement, an ultrasound medical-imaging-and-treatment transducer performs imaging and treatment at separate times. In another arrangement, an ultrasound medical-imaging transducer and a separate ultrasound medical-treatment transducer are used. The emitted ultrasound medical-treatment beam can be electronically or mechanically focused at different distances from the transducer corresponding to different treatment depths within patient tissue and/or steered to different beam angles. The transducer can have one transducer element or an array of transducer elements.

Known ultrasound medical systems and methods include deploying an end effector having an ultrasound transducer (powered by a controller) outside the body to break up kidney stones inside the body, endoscopically inserting an end effector having an ultrasound transducer in the rectum to medically destroy prostate cancer, laparoscopically inserting an end effector having an ultrasound transducer in the abdominal cavity to medically destroy a cancerous liver tumor, intravenously inserting a catheter end effector having an ultrasound transducer into a vein in the arm and moving the catheter to the heart to medically destroy diseased heart tissue, and interstitially inserting a needle end effector having an ultrasound transducer needle into the tongue to medically destroy tissue to reduce tongue volume to reduce snoring.

Still, scientists and engineers continue to seek improved ultrasound medical treatment systems and methods.

### SUMMARY OF THE INVENTION

A first expression of an embodiment of an ultrasound medical treatment system includes an ultrasound medical-treatment transducer and a controller. The controller controls the medical-treatment transducer to emit ultrasound to thermally ablate patient tissue. The control includes a control parameter. The controller changes the control parameter based on receiving an indication of an occurrence in the patient tissue of a transient, ultrasound-caused, ultrasound-attenuating effect.

A method of the invention is for medically treating patient tissue with ultrasound and includes steps a) through e). Step a) includes obtaining an ultrasound medical-treatment transducer. Step b) includes controlling the medical-treatment transducer to emit ultrasound to thermally ablate the patient tissue, wherein the control includes a control parameter, and wherein the control parameter is set to a first setting. Step c) includes receiving an indication of an occurrence in the patient tissue of a transient, ultrasound-caused, ultrasound-attenuating effect. Step d) includes changing the control parameter to a second setting based on receiving the indication. Step e) includes controlling the medical-treatment transducer to emit ultrasound to thermally ablate the patient tissue, wherein the control parameter is set to the second setting.

Several benefits and advantages are obtained from one or more of the first expression of the embodiment and/or the method of the invention. Changing a control parameter when an indication of an occurrence in the patient tissue of a transient, ultrasound-caused, ultrasound-attenuating effect has been received allows, in one example, the ultrasound acoustic power density of the medical-treatment transducer to be reduced at the onset of an ultrasound-attenuating effect caused by bubble activity from tissue cavitation and/or boiling to substantially eliminate or reduce such effect to increase the treatment depth in the patient tissue so that larger volumes of tissue can be ablated within a single treatment procedure. The use of feedback control should provide more consistent lesion size and quality across different tissue properties, geometries, and ultrasonic source conditions, and the resulting reduction of ultrasound-attenuating effects (e.g., screening and shadowing ultrasound effects) should allow the formation of more regular and controllable (and therefore more spatially selective) thermal lesions.

The present invention has, without limitation, application in conventional extracorporeal, endoscopic, laparoscopic, intra-cardiac, intravenous, interstitial and open surgical instrumentation as well as application in robotic-assisted surgery.

### BRIEF DESCRIPTION OF THE FIGURES

FIG. 1 is a schematic view of an embodiment of an ultrasound medical treatment system of the invention together with a cross section of a portion of a patient illustrated in the form of patient tissue to be thermally ablated by the system; and

FIG. 2 is a block diagram of a method of the invention for medically treating patient tissue with ultrasound which optionally can employ the embodiment of the ultrasound medical treatment system of FIG. 1.

### DETAILED DESCRIPTION OF THE INVENTION

Before explaining the present invention in detail, it should be noted that the invention is not limited in its application or use to the details of construction and arrangement of parts and/or steps illustrated in the accompanying drawings and description. The illustrative embodiment, examples, and method of the invention may be implemented or incorporated in other embodiments, examples, methods, variations and modifications, and may be practiced or carried out in various ways. Furthermore, unless otherwise indicated, the terms and expressions employed herein have been chosen for the purpose of describing the illustrative embodiment and method of the present invention for the convenience of the reader and are not for the purpose of limiting the invention.

It is understood that any one or more of the following-described method, expressions of an embodiment, examples, implementations, applications, variations, modifications, etc. can be combined with any one or more of the other following-described method, expressions of an embodiment, examples, implementations, applications, variations, modifications, etc. For example, and without limitation, the method of the invention can be performed using the embodiment of the invention.

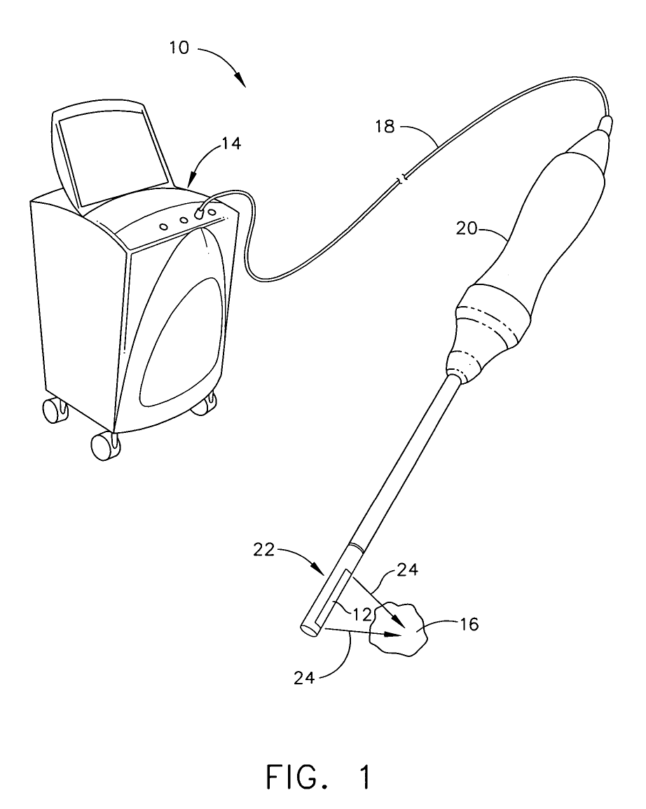

Referring now to the drawings, an embodiment of an ultrasound medical treatment system 10 is shown in FIG. 1. In a first expression of the embodiment of FIG. 1, an ultrasound medical treatment system 10 includes an ultrasound medical treatment transducer 12 and a controller 14. The controller 14 controls the medical treatment transducer 12 to emit ultrasound to thermally ablate (i.e., create a lesion in) patient tissue 16. The control includes a control parameter. The controller 14 changes the control parameter based on receiving an indication of an occurrence in the patient tissue 16 of a transient, ultrasound-caused, ultrasound-attenuating effect. In one example, the control parameter is one of a plurality of control parameters, and the controller changes one or more or all of the plurality of control parameters based on receiving an indication of an occurrence in the patient tissue 16 of at least one transient, ultrasound-caused, ultrasound-attenuating effect.

In one construction of the first expression of the embodiment of FIG. 1, a cable 18 operatively connects the controller 14 to the transducer 12. In one variation, the cable 18 connects the controller 14 to a handpiece 20 which is operatively connected to an end effector 22 which supports the transducer 12. In FIG. 1, the envelope of ultrasound (which is shown as a focused beam but can be an unfocused or divergent beam) from the transducer 12 is indicated by arrowed lines 24. In this construction, the ultrasound medical-treatment transducer 12 includes either a single ultrasound medical-treatment transducer element or an array of ultrasound medical-treatment transducer elements.

In one application of the first expression of the embodiment of FIG. 1, the control parameter is chosen from the group consisting of an ultrasonic acoustic power density of the ultrasound emitted by the medical-treatment transducer 12, an ultrasonic frequency of the ultrasound emitted by the medical-treatment transducer 12, a beam characteristic of the ultrasound emitted by the medical-treatment transducer 12, a duty cycle of the ultrasound emitted by the medical-treatment transducer 12, and a pulse sequence of the ultrasound emitted by the medical-treatment transducer 12. It is noted that the duty cycle is the ratio of the therapy on time to the total treatment time for a pulsed controller, and that the duty cycle is 1 (unity), or the pulse sequence is continuous, for a non-pulsed (continuous) controller. In one variation, the beam characteristic is chosen from the group consisting of an active aperture of the beam, a focusing characteristic (e.g., focal distance or focal area) of the beam, and a steering angle of the beam. In one modification, the medical treatment transducer 12 has an array of transducer elements, and the active aperture is the number of activated transducer elements in the array. Other control parameters, including other beam characteristics, are left to the artisan.

In one implementation of the first expression of the embodiment of FIG. 1, the ultrasound-attenuating effect is caused by at least one cause chosen from the group consisting of bubble activity from tissue cavitation, bubble activity from tissue boiling, and a temperature-related change in tissue ultrasonic absorption. Other ultrasound-attenuating effect causes are left to the artisan.

In one employment of the first expression of the embodiment of FIG. 1, the indication of the occurrence of the ultrasound-attenuating effect is based on an imaging ultrasound echo (i.e., at least one imaging ultrasound echo) from the patient tissue 16. In one example, a change in echo energy, either hyperechoicity and/or echo attenuation, indicates the occurrence of the ultrasound-attenuating effect, wherein echo attenuation would occur distal to (i.e., at a greater distance from the medical treatment transducer 12) hyperechoicity in the patient tissue 16. In one variation, feedback from an imaging ultrasound B-scan display is employed to indicate the occurrence of the ultrasound-attenuating effect. In another example, the indication of the occurrence of the ultrasound-attenuating effect is based on the (amplitude, phase, spectrum and/or waveform) difference between a first and a later-in-time second imaging ultrasound echo from the same location of patient tissue 16 (either proximal to, at, or distal to the ultrasound treatment beam focus). In a different employment, the indication of the occurrence of the ultrasound-attenuating effect is based on an MRI (magnetic resonance imaging) image of the patient tissue. Other employments, including those for non-focused and for divergent ultrasound treatment beams, are left to those skilled in the art.

In one illustration of the first expression of the embodiment of FIG. 1, the medical-treatment transducer 12 is an ultrasound medical-imaging-and-treatment transducer 26, and the imaging ultrasound echo is received by the medical-imaging-and-treatment transducer 26. In this illustration, the ultrasound medical-imaging-and-treatment transducer 26 emits low power imaging ultrasound which is reflected back from patient tissue and is received by the transducer 26 as an imaging ultrasound echo. At a different time, the transducer emits high power treatment ultrasound to ablate patient tissue.

In a second expression of the embodiment of FIG. 1, an ultrasound medical treatment system 10 includes an ultrasound medical treatment transducer 12 and a controller 14. The controller 14 controls the medical treatment transducer 12 to emit ultrasound at a first ultrasound acoustic power density to thermally ablate (i.e., create a lesion in) patient tissue 16. The controller 14 reduces the emitted ultrasound to a lower second ultrasound acoustic power density based on receiving an indication of an onset in the patient tissue 16 of a transient, ultrasound-caused, ultrasound-attenuating effect. The term “reduces” includes, without limitation, reducing to zero.

In one application of the second expression of the embodiment of FIG. 1, the lower second ultrasound acoustic power density substantially eliminates the ultrasound-attenuating effect. In another application, the lower second ultrasound acoustic power density reduces the ultrasound-attenuating effect. In one employment, the onset of the ultrasound-attenuating effect is indicated by an inception of a proximal hyperechoic region of the patient tissue with distal ultrasound attenuation. Other applications and employments are left to the artisan.

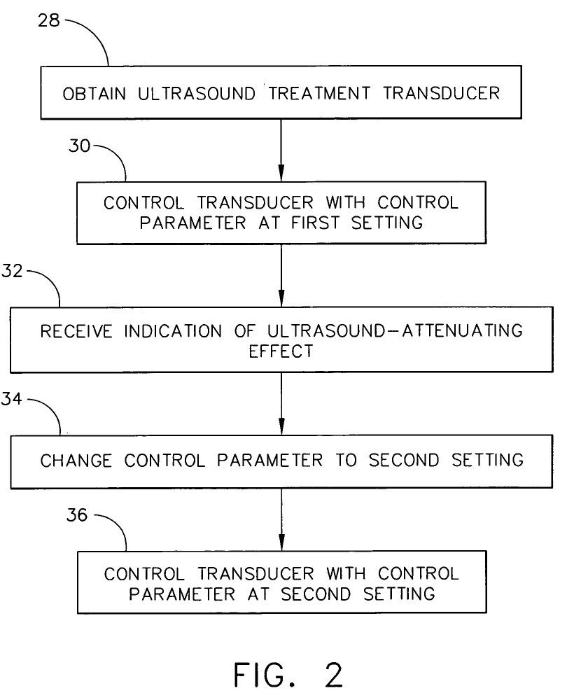

A method of the invention is shown in block diagram form in FIG. 2 and is for medically treating patient tissue 16 with ultrasound. The method includes steps a) through f). Step a) is labeled “Obtain Ultrasound Treatment Transducer” in block 28 of FIG. 2. Step a) includes obtaining an ultrasound medical-treatment transducer 12. Step b) is labeled “Control Transducer With Control Parameter At First Setting” in block 30 of FIG. 2. Step b) includes controlling the medical-treatment transducer 12 to emit ultrasound to thermally ablate the patient tissue 16, wherein the control includes a control parameter, and wherein the control parameter is set to a first setting. Step c) is labeled “Receive Indication Of Ultrasound-Attenuating Effect” in block 32 of FIG. 2. Step c) includes receiving an indication of an occurrence in the patient tissue 16 of a transient, ultrasound-caused, ultrasound-attenuating effect. Step d) is labeled “Change Control Parameter To Second Setting” in block 34 of FIG. 2. Step d) includes changing the control parameter to a second setting based on receiving the indication. It is noted that the second setting is different from the first setting. Step e) is labeled “Control Transducer With Control Parameter At Second Setting” in block 36 of FIG. 2. Step e) includes controlling the medical-treatment transducer 12 to emit ultrasound to thermally ablate the patient tissue 16, wherein the control parameter is set to the second setting.

In one example of the method of FIG. 2, a user alone in step d) effects a change in the setting of the control parameter, when the indication of step c) is received, such as by the user manually moving (translating) and/or rotating the medical-treatment transducer 12. In another example, a controller 14 controls the medical-treatment transducer 12 to emit ultrasound and the controller 14 in step d) automatically changes the setting of the control parameter, when the indication of step c) is received, such as by automatically reducing the ultrasound acoustic power density emitted by the transducer 12. In an additional example, a user in step d) changes the setting of the control parameter by changing a setting of the controller 14. Other examples are left to the artisan. Multiple changes in setting the control parameter can be employed by repeating steps c) through e) for different settings.

In one application of the method of FIG. 2, the control parameter is chosen from the group consisting of an ultrasonic acoustic power density of the ultrasound emitted by the medical-treatment transducer 12, an ultrasonic frequency of the ultrasound emitted by the medical-treatment transducer 12, a beam characteristic of the ultrasound emitted by the medical-treatment transducer 12, a duty cycle of the ultrasound emitted by the medical-treatment transducer 12, and a pulse sequence of the ultrasound emitted by the medical-treatment transducer 12. It is noted that the duty cycle is the ratio of the therapy on time to the total treatment time for a pulsed controller, and that the duty cycle is 1 (unity), or the pulse sequence is continuous, for a non-pulsed (continuous) controller. In one variation, the beam characteristic is chosen from the group consisting of an active aperture of the beam, a focusing characteristic (e.g., focal distance or focal area) of the beam, and a steering angle of the beam. In one modification, the medical treatment transducer 12 has an array of transducer elements, and the active aperture is the number of activated transducer elements in the array. Other control parameters, including other beam characteristics, are left to the artisan.

In a first enablement of the method of FIG. 2, the control parameter is an ultrasonic acoustic power density of the emitted ultrasound, wherein the second setting is lower than the first setting and substantially eliminates the ultrasound-attenuating effect or reduces the ultrasound-attenuating effect. In a second enablement, the control parameter is an ultrasonic frequency of the emitted ultrasound, wherein the second setting is lower than the first setting and substantially eliminates the ultrasound-attenuating effect or reduces the ultrasound-attenuating effect. In a third enablement, the control parameter is a duty cycle of the emitted ultrasound, wherein the second setting is lower than the first setting and substantially eliminates the ultrasound-attenuating effect or reduces the ultrasound-attenuating effect. A lower setting of an ultrasonic acoustic power density or a duty cycle includes, without limitation, a zero setting.

In a fourth enablement, the control parameter is an active aperture of the beam of emitted ultrasound, wherein the second setting is smaller than the first setting (such as by inactivating one or more or all transducer elements in a transducer having an array of transducer elements) and substantially eliminates the ultrasound-attenuating effect or reduces the ultrasound-attenuating effect. In a fifth enablement, the control parameter is a focusing characteristic (e.g., distance or focal area) of the beam of emitted ultrasound, wherein the second setting is a larger focusing characteristic (e.g., a larger focal distance or a larger focal area) and substantially eliminates the ultrasound-attenuating effect or reduces the ultrasound-attenuating effect. In a sixth enablement, the control parameter is a steering angle of the beam of emitted ultrasound, wherein the second setting is later changed to a third setting (or back to the first setting) to substantially eliminate any ultrasound-attenuating effect occurring at the second setting or to reduce any ultrasound-attenuating effect occurring at the second setting when the steering angle is at the second setting. It is noted that any of the implementations can have the second setting later return to the first setting when the ultrasound-attenuating effect at the first setting has been substantially eliminated or reduced by changing to the second setting.

In one implementation of the method of FIG. 2, the ultrasound-attenuating effect is caused by at least one cause chosen from the group consisting of bubble activity from tissue cavitation, bubble activity from tissue boiling, and a temperature-related change in tissue ultrasonic absorption. Other ultrasound-attenuating effect causes are left to the artisan.

In one employment of the method of FIG. 2, the indication of the occurrence of the ultrasound-attenuating effect is based on an imaging ultrasound echo from the patient tissue 16. In one illustration of the method of FIG. 2, the medical-treatment transducer 12 is an ultrasound medical-imaging-and-treatment transducer 26, and the imaging ultrasound echo is received by the medical-imaging-and-treatment transducer 26. In one example of the method of FIG. 2, the control parameter is an ultrasonic acoustic power density, wherein the second setting is lower than the first setting and substantially eliminates the ultrasound-attenuating effect. In one variation of this example, the onset of the ultrasound-attenuating effect is indicated by an inception of a proximal hyperechoic region of the patient tissue with distal ultrasound attenuation.

Applicants performed a first experiment on ex vivo bovine liver tissue using an ultrasound medical-imaging-and-treatment transducer having a linear array of 32 transducer elements. An active aperture of 16 elements delivered 48 watts of ultrasound acoustic power at an ultrasound acoustic power density at the source of 84 watts per square centimeter. This aperture was electronically focused at a depth of 63 millimeters. The total treatment time was one minute of which approximately 25 seconds elapsed before the appearance of a hyperechoic spot on the ultrasound echo image. The resulting maximum tissue ablation depth was about 11 millimeters. The hyperechoic spot indicated a region of tissue exhibiting an ultrasound-attenuating effect (such as bubble activity from tissue cavitation and/or boiling). The medical-treatment ultrasound beyond (distal) this region is attenuated so that thermal lesions were not created beyond 11 millimeters.

Applicants, using an example of the method of the invention, performed a second experiment on another area of the same piece of tissue with initial control parameters identical to those of the first experiment. A hyperechoic spot appeared on the ultrasound echo image after 35 seconds of treatment. At this point, the ultrasound acoustic power density at the source was reduced from 84 watts per square centimeter to 55 watts per square centimeter for the remainder of the one minute treatment. The resulting maximum tissue ablation depth was about 18 millimeters which was significantly greater than that achieved in the first experiment. It is noted that this increased treatment depth occurred even though less total thermal energy was delivered in the second experiment than in the first experiment. The treated tissue area of the second experiment incurred much less over-treatment and cracking than did the treated tissue area of the first experiment.

Several benefits and advantages are obtained from one or more of the expressions of the embodiment and/or the method of the invention. Changing a control parameter when an indication of an occurrence in the patient tissue of a transient, ultrasound-caused, ultrasound-attenuating effect has been received allows, in one example, the ultrasound acoustic power density of the medical-treatment transducer to be reduced at the onset of an ultrasound-attenuating effect caused by bubble activity from tissue cavitation and/or boiling to substantially eliminate or reduce such effect to increase the treatment depth in the patient tissue so that larger volumes of tissue can be ablated within a single treatment procedure. The use of feedback control should provide more consistent lesion size and quality across different tissue properties, geometries, and ultrasonic source conditions, and the resulting reduction of ultrasound-attenuating effects (e.g., screening and shadowing ultrasound effects) should allow the formation of more regular and controllable (and therefore more spatially selective) thermal lesions.

While the present invention has been illustrated by a description of a method and several expressions of an embodiment, it is not the intention of the applicants to restrict or limit the spirit and scope of the appended claims to such detail. Numerous other variations, changes, and substitutions will occur to those skilled in the art without departing from the scope of the invention. For instance, the ultrasound method and system embodiment of the invention have application in robotic assisted surgery taking into account the obvious modifications of such method, system embodiment and components to be compatible with such a robotic system. It will be understood that the foregoing description is provided by way of example, and that other modifications may occur to those skilled in the art without departing from the scope and spirit of the appended Claims.

1. An ultrasound medical treatment system comprising:
a) an ultrasound medical-treatment transducer; and
b) a controller which controls the medical-treatment transducer to emit ultrasound to thermally ablate patient tissue, wherein the control includes a control parameter, and wherein the controller changes the control parameter based on receiving an indication of an occurrence in the patient tissue of a transient, ultrasound-caused, ultrasound-attenuating effect.

2. The ultrasound medical treatment system of claim 1, wherein the control parameter is chosen from the group consisting of an ultrasonic acoustic power density of the ultrasound emitted by the medical-treatment transducer, an ultrasonic frequency of the ultrasound emitted by the medical-treatment transducer, a beam characteristic of the ultrasound emitted by the medical-treatment transducer, a duty cycle of the ultrasound emitted by the medical-treatment transducer, and a pulse sequence of the ultrasound emitted by the medical-treatment transducer.

3. The ultrasound medical treatment system of claim 2, wherein the beam characteristic is chosen from the group consisting of an active aperture of the beam, a focusing characteristic of the beam, and a steering angle of the beam.

4. The ultrasound medical treatment system of claim 2, wherein the ultrasound-attenuating effect is caused by at least one cause chosen from the group consisting of bubble activity from tissue cavitation, bubble activity from tissue boiling, and a temperature-related change in tissue ultrasonic absorption.

5. The ultrasound medical treatment system of claim 4, wherein the indication of the occurrence of the ultrasound-attenuating effect is based on an imaging ultrasound echo from the patient tissue.

6. The ultrasound medical treatment system of claim 5, wherein the medical-treatment transducer is an ultrasound medical-imaging-and-treatment transducer, and wherein the imaging ultrasound echo is received by the medical-imaging-and-treatment transducer.

7. The ultrasound medical treatment system of claim 1, wherein the ultrasound-attenuating effect is caused by at least one cause chosen from the group consisting of bubble activity from tissue cavitation, bubble activity from tissue boiling, and a temperature-related change in tissue ultrasonic absorption.

8. The ultrasound medical treatment system of claim 1, wherein the indication of the occurrence of the ultrasound-attenuating effect is based on an imaging ultrasound echo from the patient tissue.

9. An ultrasound medical treatment system comprising:
a) an ultrasound medical-treatment transducer; and
b) a controller which controls the medical-treatment transducer to emit ultrasound at a first ultrasound acoustic power density to thermally ablate patient tissue, wherein the controller reduces the emitted ultrasound to a lower second ultrasound acoustic power density based on receiving an indication of an onset in the patient tissue of a transient, ultrasound-caused, ultrasound-attenuating effect.

10. The ultrasound medical treatment system of claim 9, wherein the lower second ultrasound acoustic power density substantially eliminates the ultrasound-attenuating effect.

11. The ultrasound medical treatment system of claim 10, wherein the onset of the ultrasound-attenuating effect is indicated by an inception of a proximal hyperechoic region of the patient tissue with distal ultrasound attenuation.

12. A method for medically treating patient tissue with ultrasound comprising the steps of:
a) obtaining an ultrasound medical-treatment transducer;
b) controlling the medical-treatment transducer to emit ultrasound to thermally ablate the patient tissue, wherein the control includes a control parameter, and wherein the control parameter is set to a first setting;
c) receiving an indication of an occurrence in the patient tissue of a transient, ultrasound-caused, ultrasound-attenuating effect;
e) changing the control parameter to a second setting based on receiving the indication; and
f) controlling the medical-treatment transducer to emit ultrasound to thermally ablate the patient tissue, wherein the control parameter is set to the second setting.

13. The method of claim 12, wherein the control parameter is chosen from the group consisting of an ultrasonic acoustic power density of the ultrasound emitted by the medical-treatment transducer, an ultrasonic frequency of the ultrasound emitted by the medical-treatment transducer, a beam characteristic of the ultrasound emitted by the medical-treatment transducer, a duty cycle of the ultrasound emitted by the medical-treatment transducer, and a pulse sequence of the ultrasound emitted by the medical-treatment transducer.

14. The method of claim 13, wherein the beam characteristic is chosen from the group consisting of an active aperture of the beam, a focusing characteristic of the beam, and a steering angle of the beam.

15. The method of claim 13, wherein the ultrasound-attenuating effect is caused by at least one cause chosen from the group consisting of bubble activity from tissue cavitation, bubble activity from tissue boiling, and a temperature-related change in tissue ultrasonic absorption.

16. The method of claim 15, wherein the indication of the occurrence of the ultrasound-attenuating effect is based on an imaging ultrasound echo from the patient tissue.

17. The method of claim 16, wherein the medical-treatment transducer is an ultrasound medical-imaging-and-treatment transducer, and wherein the imaging ultrasound echo is received by the medical-imaging-and-treatment transducer.

18. The method of claim 12, wherein the ultrasound-attenuating effect is caused by at least one cause chosen from the group consisting of bubble activity from tissue cavitation, bubble activity from tissue boiling, and a temperature-related change in tissue ultrasonic absorption.

19. The method of claim 12, wherein the indication of the occurrence of the ultrasound-attenuating effect is based on an imaging ultrasound echo from the patient tissue.

20. The method of claim 12, wherein the control parameter is an ultrasonic acoustic power density, wherein the second setting is lower than the first setting and substantially eliminates the ultrasound-attenuating effect, and wherein the onset of the ultrasound-attenuating effect is indicated by an inception of a proximal hyperechoic region of the patient tissue with distal ultrasound attenuation.
